(19) United States
(12) Patent Application Publication
(10) Pub. No.: US 2023/0381764 A1
(43) Pub. Date: Nov. 30, 2023

(54) USE OF HIGHLY ISOSELECTIVE, THERMALLY STABLE FERROCENE CATALYSTS FOR PROPYLENE HYDROFORMYLATION

(71) Applicant: Eastman Chemical Company, Kingsport, TN (US)

(72) Inventors: Mesfin Ejerssa Janka, Kingsport, TN (US); Stephanie Rollins Testerman, Kingsport, TN (US); Jody Lee Rodgers, Gilmer, TX (US); Michelle Nicole Tuttle, Hallsville, TX (US)

(73) Assignee: Eastman Chemical Company, Kingsport, TN (US)

(21) Appl. No.: 18/245,906

(22) PCT Filed: Sep. 22, 2021
(86) PCT No.: PCT/US2021/051404

§ 371 Date: Mar. 20, 2023

Related U.S. Application Data
(60) Provisional application No. 63/085,239, filed on Sep. 30, 2020.

(51) Int. Cl. B01J 31/24; B01J 23/46; C07C 45/50

(52) U.S. Cl. CPC B01J 31/2409; B01J 23/464; C07C 45/50; B01J 2231/321; C07C 2531/24; C07C 2523/46

(57) ABSTRACT

Processes for preparing aldehydes from olefins under hydroformylation temperature and pressure conditions are disclosed. The processes include a step of contacting at least one olefin with hydrogen and carbon monoxide in the presence of at least one solvent and a transition metal-based catalyst composition comprising a ferrocene-based biphosphine ligand.

### BACKGROUND OF INVENTION

The hydroformylation reaction, also known as the oxo reaction, is used extensively in commercial processes for the preparation of aldehydes by the reaction of one mole of an olefin with one mole each of hydrogen and carbon monoxide. A particularly important use of the reaction is in the preparation of normal (n-) and iso (iso-) butyraldehydes from propylene. Both products are key building blocks for the synthesis of many chemical intermediates like alcohols, carboxylic acids, esters, plasticizers, glycols, essential amino acids, flavorings, fragrances, polymers, insecticides, hydraulic fluids, and lubricants.

At present, high n-selectivity is more easily achieved whereas achievement of high iso-selectivity remains challenging. Different approaches have been attempted throughout the years to tackle this problem, including the use of various ligands (Phillips, Devon, Puckette, Stavinoha, Vanderbilt, (Eastman Kodak Company, U.S. Pat. No. 4,760,194) and carrying out reactions under aqueous conditions (Riisager, Eriksen, Hjorkjmr, Fehrmann, J. Mol. Catal. A: Chem. 2003, 193, 259). The results have not been entirely satisfactory, with either unimpressive iso-selectivity and/or because the reaction needs to be run at an undesirable temperature

U.S. Pat. No. 5,371,256 discloses ferrocenyl diphosphines as ligands for homogeneous catalysts. They are reported to be useful as enantioselective hydrogenation catalysts for the homogeneous hydrogenation of prochiral unsaturated compounds.

U.S. Pat. No. 9,308,527 describes bidentate ferrocene-linked phosphine-phosphoramidate compounds that exhibit iso-selectivity. Hydroformylation catalyst compositions and methods of hydroformylation using the compounds are also disclosed. Methods of making the compounds are also disclosed.

An iso-selectivity of 63% was reported in a reaction carried out at 19° C. (Norman, Reek, Besset, (Eastman Chemical Company), U.S. Pat. No. 8,710,275). However, in some instances this is not desirable because hydroformylation reactions conducted at lower temperatures may result in lower reaction rates, so carrying out the reaction at a higher temperature is generally preferred in industry. In this case, the iso-selectivity was reduced to 38% when the reaction was carried out at 80° C.

U.S. Pat. No. 10,144,751 discloses ligands for use with catalyst compositions used in hydroformylation reactions that may be used with various octofluorotoluene or hydrocarbon solvents to achieve an increase in TON with an increase in temperature, and/or show isoselectivity that is surprisingly high in comparison to hydroformylation reactions using common solvents.

U.S. Pat. No. 10,183,961 discloses ligands for use with catalyst compositions used in hydroformylation reactions that may be used with various ester solvents to achieve an increase in TON with an increase in temperature, and/or show isoselectivity that is surprisingly high in comparison to the hydroformylation reactions using common solvents.

There remains a need for catalysts showing iso-selectivity for propylene hydroformylation, methods of making them, and mixtures thereof.

### SUMMARY OF INVENTION

According to an embodiment, the disclosure teaches a process for preparing at least one aldehyde under hydroformylation temperature and pressure conditions. The process includes contacting at least one olefin, which in some embodiments may be propylene, with hydrogen and carbon monoxide in the presence of at least one solvent and a transition metal-based catalyst composition, which in some embodiments may be rhodium based, that includes a ferrocene-based biphosphine ligand. The ligand is represented by the following general formula A and its mirror images:

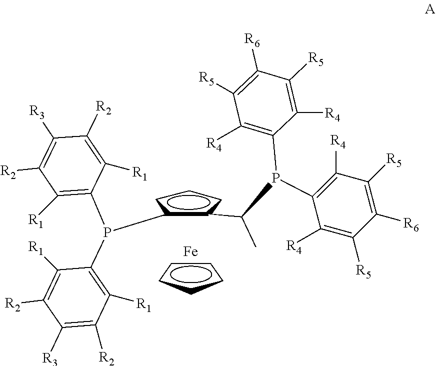

Wherein:
R1, R2, R3, R4, R5 and R6 are independently selected from H, F, CI, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent.

In another aspect, the process includes contacting at least one olefin, which in some embodiments may be propylene, with hydrogen and carbon monoxide in the presence of at least one solvent and a transition metal-based catalyst composition, which in some embodiments may be rhodium based, that includes a mixture of ferrocene-based biphosphine ligands. The ligands are represented by the following general formulas A and B and their mirror images:

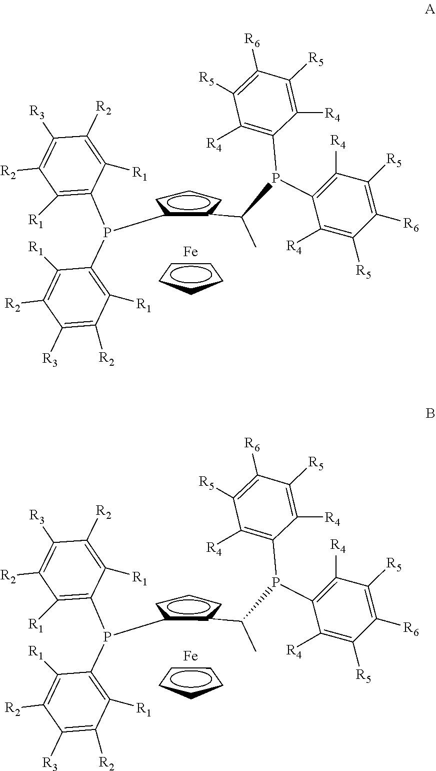

wherein:

R1, R2, R3, R4, R5 and R6 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent.

These ligands in the presence of Rh metal show very good isoselectivity for hydroformylation of propylene. We have also found out that hydroformylation of propylene using mixed ligands A and B gave the same isoselectivity as enantiopure ligand A. This will cut back two steps from the ligand manufacturing process. In addition, these ligands show very good stability at high temperature and the selectivity can be varied by varying ligand to Rh ratio.

The aldehyde product of the process comprising an iso-selectivity in some embodiments of about 55% to about 70%, about 56% to about 68%, about 57 to about 67%, or about 55% or greater, or 57% or greater

In addition, the process operates in a pressure range in some embodiments of about 2 atm to about 80 atm, about 5 to about 70 atm, about 8 atm to about 20 atm, about 8 atm, or about 20 atm. The process also operates in a temperature range in some embodiments of about 40 to about 150 degrees Celsius, about 40 about 120 degrees Celsius, about 40 to about 100 degrees Celsius, about 50 about 90 degrees Celsius, about 50 degrees Celsius, about 75 degrees Celsius, or about 90 degrees Celsius.

### BRIEF DESCRIPTION OF THE DRAWINGS

FIGS. 1a to 1d illustrate the results of 4 days' continuous hydroformylation of propylene using a ligand of the present invention.

### DETAILED DESCRIPTION

In one aspect, the invention relates to processes for preparing at least one aldehyde under hydroformylation temperature and pressure conditions. The process includes contacting at least one olefin, which may be propylene, with hydrogen and carbon monoxide in the presence of at least one solvent and a transition metal-based catalyst composition, which in some embodiments may be rhodium based, that includes a ferrocene-based biphosphine ligand. The ligand is represented by the following general formula A and its mirror images:

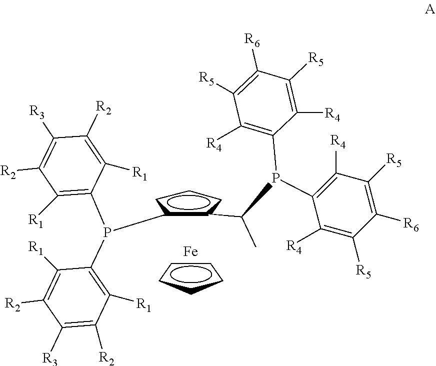

In another aspect, the invention relates to processes that include contacting at least one olefin, which in some embodiments may be propylene, with hydrogen and carbon monoxide in the presence of at least one solvent and a transition metal-based catalyst composition, which in some embodiments may be rhodium based, that includes a mixture of ferrocene-based biphosphine ligands. The ligands are represented by the following general formulas A and B, and their mirror images:

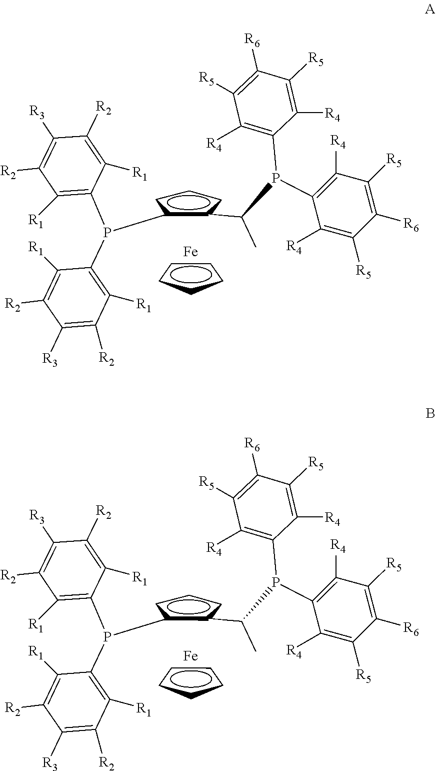

In both these aspects, R1, R2, R3, R4, R5 and R6 may be independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent.

In a further aspect, R1, R2, R3, R4, R5 and R6 may be independently selected from H, or C1 to C3 alkyl.

In a further aspect, R1, R2, R3, R4 and R6 may be hydrogen, and R5 may be methyl or ethyl.

In a further aspect, R1, R2, R4, R5, and R6 may be hydrogen, and R3 may be H, F, Cl, or Br, and especially F.

In a further aspect, R1, R2, R3, R4, and R5 may be hydrogen, and R6 may be H, F, Cl, or Br, and especially F.

In a further aspect, R1, R2, R4, and R5 may be hydrogen, and R3 and R6 may be H, F, Cl, or Br, and especially F

In yet another aspect, R1, R2, R3, R4, R5 R6 are hydrogen.

Specific formulations may include the following ligands, including specific stereoisomers and mixtures thereof, and their mirror images:

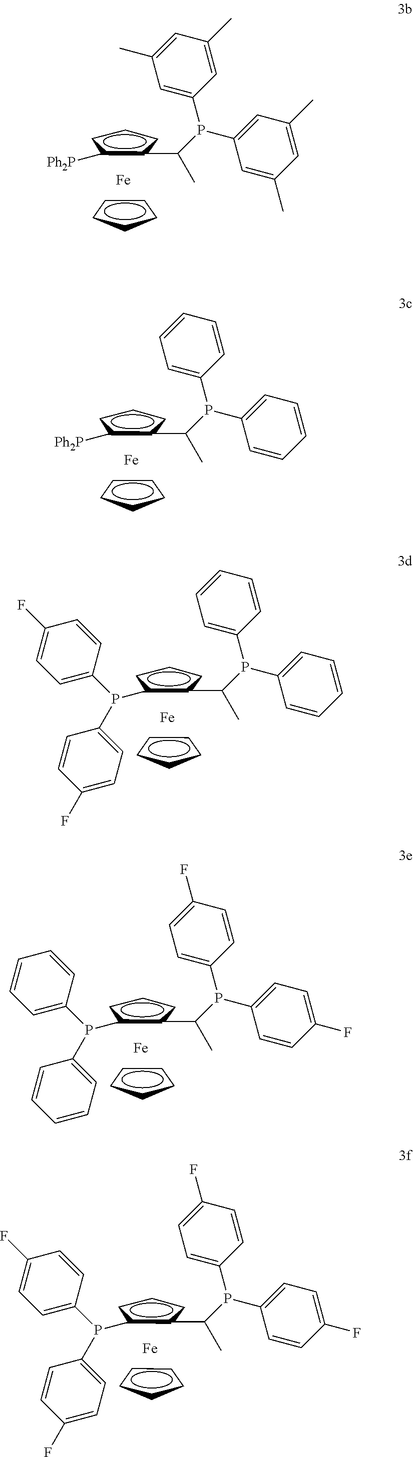

The mixtures useful according to the invention may be prepared using processes set out in the examples. U.S. Pat. Appln. No. 63/085,241, filed herewith and having common assignee, is directed to these inventive processes for preparing these mixtures.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, each numerical parameter should at least be construed in light of the number of reported significant digits and by applying ordinary rounding techniques. Further, the ranges stated in this disclosure and the claims are intended to include the entire range specifically and not just the endpoint(s). For example, a range stated to be 0 to 10 is intended to disclose all whole numbers between 0 and 10 such as, for example 1, 2, 3, 4, etc., all fractional numbers between 0 and 10, for example 1.5, 2.3, 4.57, 6.1113, etc., and the endpoints 0 and 10.

Notwithstanding that the numerical ranges and parameters setting forth the broad scope of the invention are approximations, the numerical values set forth in the specific examples are intended to be reported precisely in view of methods of measurement. Any numerical value, however, inherently contains certain errors necessarily resulting from the standard deviation found in their respective testing measurements.

It is to be understood that the mention of one or more process steps does not preclude the presence of additional process steps before or after the combined recited steps or intervening process steps between those steps expressly identified. Moreover, the denomination of process steps, ingredients, or other aspects of the information disclosed or claimed in the application with letters, numbers, or the like is a convenient means for identifying discrete activities or ingredients and the recited lettering can be arranged in any sequence, unless otherwise indicated.

As used herein, the singular forms “a”, “an”, and “the” include plural referents unless the context clearly dictates otherwise. For example, reference to a Cn alcohol equivalent is intended to include multiple types of Cn alcohol equivalents. Thus, even use of language such as “at least one” or “at least some” in one location is not intended to imply that other uses of “a”, “an”, and “the” excludes plural referents unless the context clearly dictates otherwise. Similarly, use of the language such as “at least some” in one location is not intended to imply that the absence of such language in other places implies that “all” is intended, unless the context clearly dictates otherwise.

As used herein the term “and/or”, when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

The term “catalyst”, as used herein, has its typical meaning to one skilled in the art as a substance that increases the rate of chemical reactions without being consumed by the reaction in substantial amounts.

The term “alkyl” as used herein refers to a group containing one or more saturated carbons, such as methyl, ethyl, n-propyl, isopropyl, n-butyl, isobutyl, tert-butyl, n-pentyl, n-hexyl, 2-ethylhexyl, n-octyl, n-decyl, dodecyl, n-octadecyl and various isomers thereof. Unless specifically indicated otherwise, “alkyl” includes linear alkyl, branched alkyl, and cycloalkyl groups. A “linear alkyl group” refers to an alkyl group having no branching of carbon atoms. A “branched alkyl group” refers to an alkyl group having branching of carbon atoms such that at least one of the carbons in the group is bonded to at least three other atoms that are either carbons within that group or atoms outside the group. Thus, “an alkyl group having branching at the alpha carbon” is a type of branched alkyl group in which a carbon that is bonded to two carbons within the alkyl group is also bonded to a third (non-hydrogen) atom not located within the alkyl group. A “cycloalkyl” or “cyclic alkyl” group is an alkyl group that is arranged in a ring of alkyl carbons, such as a cyclopentyl or a cyclohexyl group.

The term “aryl” as used herein refers to a group that is or contains an aromatic ring containing carbons. Some examples of aryl groups include phenyl and naphthyl groups.

The term “aryloxy” as used herein refers to a group having the structure shown by the formula —O—Ar, wherein Ar is an aryl group as described above.

The term “aralkyl” used herein refers to an aryl group in which an alkyl group is substituted for at least one of the hydrogens.

The term “alkaryl” used herein refers to an alkyl group in which an aryl group is substituted for at least one of the hydrogens.

The term “aryldialkylsilyl” refers to a group in which a single silicon atom is bonded to two alkyl groups and one aryl group.

The term “diarylalkylsilyl” refers to a group in which a single silicon atom is bonded to one alkyl group and two aryl group.

The term “phenyl” refers to an aryl substituent that has the formula C₆H₅, provided that a “substituted phenyl” has one or more group substituted for one or more of the hydrogen atoms.

The term “trialkylsilyl” refers to a group in which three alkyl groups are bonded to the same silicon atom.

The term “triarylsilyl” refers to a group in which three aryl groups are bonded to the same silicon atom.

The process described herein thus provides that an olefin is contacted with hydrogen and carbon monoxide in the presence of a transition metal catalyst and ligand. In an embodiment, the olefin is propylene. It is also contemplated that additional olefins, such as, for example, butene, pentene, hexene, heptene, and octene could work in the process.

These ligands in the presence of Rh metal show very good isoselectivity for hydroformylation of propylene. We have also found out that hydroformylation of propylene using mixed ligands B and C gave the same isoselectivity as enantiopure ligand A. In addition, these ligands show very good stability at high temperature and the selectivity can be varied by varying ligand to Rh ratio.

Thus, in one aspect, in terms of stability at high temperature, the inventive ligands of the invention may show stability at temperatures, for example, of about 85° to about 125° C., or from 90° C. to 120° C., or from 95° to 115° C.

In another aspect according to the invention, the selectivity can be varied by varying the ligand to Rh ratio. Thus, in one aspect the ligand to Rh ratio may be from about 1:1 to about 50:1, or from 2:1 to 40:1, or from 3:1 to 4:1 to 20:1, in each case based on mole ratio of ligand to rhodium.

The resultant catalyst composition of the process contains a transition metal as well ligands as described herein. In some embodiments, the transition metal catalyst contains rhodium.

Acceptable forms of rhodium include rhodium (II) or rhodium (III) salts of carboxylic acids, rhodium carbonyl species, and rhodium organophosphine complexes. Some examples of rhodium (II) or rhodium (III) salts of carboxylic acids include di-rhodium tetraacetate dihydrate, rhodium(II) acetate, rhodium(II) isobutyrate, rhodium(II) 2-ethylhexanoate, rhodium(II) benzoate and rhodium(II) octanoate. Some examples of rhodium carbonyl species include [Rh(acac)(CO)₂], Rh₄(CO)₁₂, and Rh₆(CO)₁₆. An example of rhodium organophosphine complexes is tris(triphenylphosphine) rhodium carbonyl hydride may be used.

The absolute concentration of the transition metal in the reaction mixture or solution may vary from about 1 mg/liter up to about 5000 mg/liter; in some embodiments, it is higher than about 5000 mg/liter. In some embodiments of this invention, the concentration of transition metal in the reaction solution is in the range of from about 20 to about 300 mg/liter. Ratio of moles ligand to moles of transition metal can vary over a wide range, e.g., moles of ligand:moles of transition metal ratio of from about 0.1:1 to about 500:1 or from about 0.5:1 to about 500:1. For rhodium-containing catalyst systems, the moles of ligand:moles of rhodium ratio in some embodiments is in the range of from about 0.1:1 to about 200:1 with ratios in some embodiments in the range of from about 1:1 to about 100:1, or from about 1:1 to about 10:1.

In some embodiments, catalyst is formed in situ from a transition metal compound such as [Rh(acac)(CO)₂] and a ligand. It is appreciated by those skilled in the art that a wide variety of Rh species will form the same active catalyst when contacted with ligand, hydrogen and carbon monoxide, and thus there is no limitation on the choice of Rh pre-catalyst.

In additional embodiments, the process is carried out in the presence of at least one solvent. Where present, the solvent or solvents may be any compound or combination of compounds that does not unacceptably affect the hydroformylation process and/or which are inert with respect to the catalyst, propylene, hydrogen and carbon monoxide feeds as well as the hydroformylation products. These solvents may be selected from a wide variety of compounds, combinations of compounds, or materials that are liquid under the reaction conditions at which the process is being operated. Such compounds and materials include various alkanes, cycloalkanes, alkenes, cycloalkenes, carbocyclic aromatic compounds, alcohols, carboxylic acid esters, ketones, acetals, ethers and water. Specific examples of such solvents include alkane and cycloalkanes such as dodecane, decalin, hexane, octane, isooctane mixtures, cyclohexane, cyclooctane, cyclododecane, methylcyclohexane; aromatic hydrocarbons such as benzene, toluene, xylene isomers, tetralin, cumene, alkyl-substituted aromatic compounds such as the isomers of diisopropylbenzene, triisopropylbenzene and tert-butylbenzene; alkenes and cycloalkenes such as 1,7-octadiene, dicyclopentadiene, 1,5-cyclooctadiene, octene-1, octene-2,4-vinylcyclohexene, cyclohexene, 1,5,9-cyclododecatriene, 1-pentene; crude hydrocarbon mixtures such as naphtha, mineral oils and kerosene; carboxylic acid esters such as ethyl acetate and high-boiling esters such as 2,2,4-trimethyl-1,3-pentanediol diisobutyrate as well as trimeric aldehyde ester-alcohols such as 2,2,4-trimethyl-1,3-pentanediol mono(2-methylpropanoate). The aldehyde product of the hydroformylation process also may be used.

In some embodiments, the preferred solvent is the higher boiling by-products that are naturally formed during the process of the hydroformylation reaction and the subsequent steps, e.g., distillations, that may be used for aldehyde product isolation. In some embodiments involving more volatile aldehydes, the solvent has a sufficiently high boiling to remain, for the most part, in a gas sparged reactor. Some examples of solvents and solvent combinations that may be used in the production of less volatile and non-volatile aldehyde products include 1-methyl-2-pyrrolidinone, dimethyl-formamide, perfluorinated solvents such as perfluoro-kerosene, sulfolane, water, and high boiling hydrocarbon liquids as well as combinations of these solvents.

In other aspects, regardless of ligand used, the process may use either fluorinated solvents which can be octofluorotoluene, or perfluorophenyl octyl ether or a hydrocarbon solvent which can be n-nonane, n-decane, n-undecane, or n-dodecane. It is also contemplated that other solvents may be used in combination with these solvents. In other aspects, the process may use at least one ester solvent which can be ethyl acetate, butyl butyrate, pentyl pentanoate, propyl propionate and dioctyl terephthalate.

The disclosure further provides methods for the synthesis methods as generally described here and specifically described in the Examples below.

As for formulating the catalyst systems, no special or unusual techniques are required for preparing the catalyst systems and solutions of the present invention, although in some embodiments higher activity may be observed if all manipulations of the rhodium and ligand components are carried out under an inert atmosphere, e.g., nitrogen, argon and the like. Furthermore, in some embodiments it may be advantageous to dissolve the ligand and the transition metal together in a solvent to allow complexation of the ligand and transition metal followed by crystallization of the metal ligand complex as described in U.S. Pat. No. 9,308,527 which is herein incorporated by reference in its entirety.

Appropriate reaction conditions for effective hydroformylation conditions can be used as detailed in this paragraph. In some embodiments, the process is carried out at temperatures in the range of from about 40 to about 150 degrees Celsius, about 40 to about 120 degrees Celsius, about 40 to about 100 degrees Celsius, about 50 to about 90 degrees Celsius, about 50 degrees Celsius, about 75 degrees Celsius, or about 90 degrees Celsius. In some embodiments, the total reaction pressure may range from about 2 atm to about atm, about 5 to about 70 atm, about 8 atm to about 20 atm, be about 8 atm, or be about 20 atm.

In some embodiments, the hydrogen:carbon monoxide mole ratio in the reactor may vary considerably ranging from about 10:1 to about 1:10 and the sum of the absolute partial pressures of hydrogen and carbon monoxide may range from about 0.3 to about 36 atm. In some embodiments, the partial pressure of hydrogen and carbon monoxide in the reactor is maintained within the range of from about 1 to about 14 atm for each gas. In some embodiments, the partial pressure of carbon monoxide in the reactor is maintained within the range of from about 1 to about 14 atm and is varied independently of the hydrogen partial pressure. The molar ratio of hydrogen to carbon monoxide can be varied widely within these partial pressure ranges for the hydrogen and carbon monoxide. The ratios of the hydrogen to carbon monoxide and the partial pressure of each in the synthesis gas (syngas—carbon monoxide and hydrogen) can be readily changed by the addition of either hydrogen or carbon monoxide to the syngas stream.

The amount of olefin present in the reaction mixture also is not critical. In some embodiments of the hydroformylation of propylene, the partial pressures in the vapor space in the reactor are in the range of from about 0.07 to about 35 atm. In some embodiments involving the hydroformylation of propylene, the partial pressure of propylene is greater than about 1.4 atm, e.g., from about 1.4 to about 10 atm. In some embodiments of propylene hydroformylation, the partial pressure of propylene in the reactor is greater than about 0.14 atm.

Any effective hydroformylation reactor designs or configurations may be used in carrying out the process provided by the present invention. Thus, a gas-sparged, liquid overflow reactor or vapor take-off reactor design as disclosed in the examples set forth herein may be used. In some embodiments of this mode of operation, the catalyst which is dissolved in a high boiling organic solvent under pressure does not leave the reaction zone with the aldehyde product taken overhead by the unreacted gases. The overhead gases then are chilled in a vapor/liquid separator to condense the aldehyde product and the gases can be recycled to the reactor. The liquid product is let down to atmospheric pressure for separation and purification by conventional technique. The process also may be practiced in a batchwise manner by contacting propylene, hydrogen and carbon monoxide with the present catalyst in an autoclave.

A reactor design where catalyst and feedstock are pumped into a reactor and allowed to overflow with product aldehyde, i.e. liquid overflow reactor design, is also suitable. In some embodiments, the aldehyde product may be separated from the catalyst by conventional means such as by distillation or extraction and the catalyst then recycled back to the reactor. Water soluble aldehyde products can be separated from the catalyst by extraction techniques. A trickle-bed reactor design also is suitable for this process. It will be apparent to those skilled in the art that other reactor schemes may be used with this invention.

For continuously operating reactors, it may be desirable to add supplementary amounts of the ligand (compound) over time to replace those materials lost by oxidation or other processes. This can be done by dissolving the ligand into a solvent and pumping it into the reactor as needed. The solvents that may be used include compounds that are found in the process such as olefin, the product aldehydes, condensation products derived from the aldehydes, and other esters and alcohols that can be readily formed from the product aldehydes. Example solvents include butyraldehyde, isobutyraldehyde, propionaldehyde, 2-ethylhexanal, 2-ethylhexanol, n-butanol, isobutanol, isobutyl isobutyrate, isobutyl acetate, butyl butyrate, butyl acetate, 2,2,4-trimethylpentane-1,3-diol diisobutyrate, and n-butyl 2-ethylhexanoate. Ketones such as cyclohexanone, methyl isobutyl ketone, methyl ethyl ketone, diisopropylketone, and 2-octanone may also be used as well as trimeric aldehyde ester-alcohols such as Texanol™ ester alcohol (2,2,4-trimethyl-1,3-pentanediol mono(2-methylpropanoate)).

In some embodiments, the reagents employed for the invention hydroformylation process are substantially free of materials which may reduce catalyst activity or completely deactivate the catalyst. In some embodiments, materials such as conjugated dienes, acetylenes, mercaptans, mineral acids, halogenated organic compounds, and free oxygen are excluded from the reaction.

This invention can be further illustrated by the following examples of embodiments thereof, although it will be understood that these examples are included merely for purposes of illustration and are not intended to limit the scope of the invention unless otherwise specifically indicated.

### EXAMPLES

General: All solvents, chlorodiphenylphosphine, diphenylphosphine, chlorobis(4-fluorophenyl)phosphine, bis(3,5-dimethyl-phenyl)phosphine ((HP(3,5-xyl)₂) were purchased from Aldrich Chemical Company and were used as received. Dicarbonylacetylacetonato rhodium (I) (RhAcAc(CO)₂), (R)-(−)-1-[(S)-2-(diphenylphosphino)ferrocenyl]ethyldi-3,5-xylylphosphine (3a) were received from Strem Chemicals, Inc. and were used as received. Racemic N,N-dimethyl-1-ferrocenylethylamine 1 was synthesized according to Gokel, G. W.; Ugi, I. K.; J. Chem. Edu. 1972, 49, 294-296. Bis(4-fluorophenyl)phosphine was synthesized according to Carl A. Busacca; Jon C. Lorenz; Nelu Grinberg; Nizar Haddad; Matt Hrapchak; Bachir Latli; Heewon Lee; Paul Sabila; Anjan Saha; Max Sarvestani; Sherry Shen; Richard Varsolona; Xudong; Chris H. Senanayake; Org. Lett. 2005, 7 (19), 4277.

NMR spectra were recorded on a Bruker Advance 500 MHz instrument. Proton chemical shifts are referenced to internal residual solvent protons. Signal multiplicities are given as s (singlet), d (doublet), t (triplet), q (quartet), m (multiplet), br.s (broad singlet) or a combination of the above. Where appropriate, coupling constants (J) are quoted in Hz and are reported to the nearest 0.1 Hz. All spectra were recorded at room temperature and the solvent for a spectrum is given in parentheses. NMR of compounds containing phosphorus were recorded under an inert atmosphere in dry and degassed solvent.

Ligands synthesis: It was reported in literature that Josiphos ligand 3a was synthesized in 4 steps from racemic ferrocenylethyl amine for asymmetric hydrogenation reaction. The original synthesis of Josiphos ligands, Scheme I, was described in U.S. Pat. No. 5,371,256. One of the key steps involves separation of enantiopure species 1a by reacting racemic N,N-dimethyl-1-ferrocenylethylamine with (R)(+) tartaric acid, see for example Gokel, G. W.; Ugi, I. K.; J. Chem. Edu. 1972, 49, 294-296:

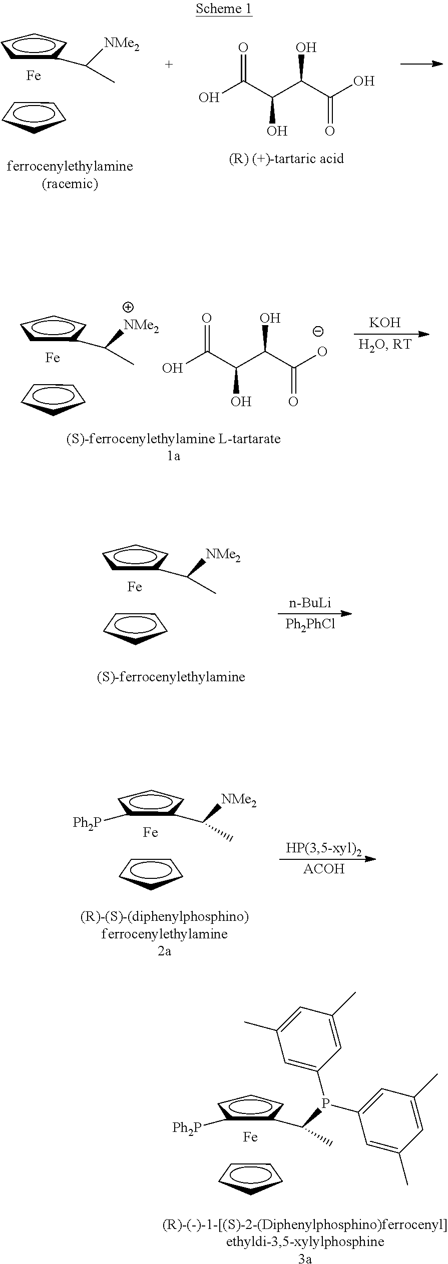

According to the inventive process claimed in our copending application filed herewith, mixed ligands 3b to 3f were synthesized from racemic N,N-dimethyl-1-ferrocenylethylamine in only two steps, Scheme II:

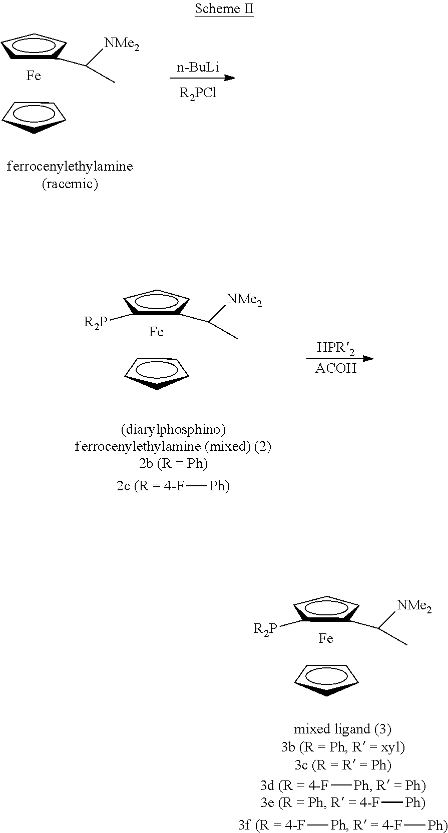

### Example 1: Preparation of diphenylphosphino)ferrocenylethylamine 2b (mixed)

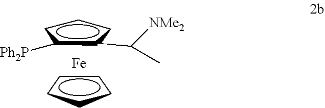

In a dry box, a 500 mL round bottom flask was charged with 5.00 g of racemic N,N-dimethyl-1-ferrocenylethylamine 1 (18.67 mmol) and 50 mL tert-butyl methyl ether (TBME). n-Butyllithium (8.96 mL, 22.4 mmol, 1.2 eq.) was added via an addition funnel over 1 hr while maintaining temperature below 25° C. The addition funnel was rinsed with 20 mL of TBME into the flask. The mixture was stirred at ambient temperature for 1 hr. Chlorodiphenylphosphine (5.15 g, 22.4 mmol, 1.2 eq.) in 20 mL TBME was added dropwise into the flask over 30 min, maintaining temperature less than 35° C. The mixture was stirred under reflux for 2.5 hrs and then at ambient temperature for 1.5 hrs. The mixture was cooled in an ice-water bath and the reaction was quenched with 100 mL saturated aqueous sodium bicarbonate, while maintaining temperature below 15° C., followed by 150 mL water. The layers were separated, and the aqueous layer was extracted with toluene (3×200 mL). The combined organic layers were dried with sodium sulfate and concentrated to afford 9.57 g crude product as a brown oil. The crude product was crystallized from refluxing alcohol by cooling to 4° C. overnight. The precipitated, orange solid, was filtered off and washed with 50 mL cold ethanol at 0° C. yielding 5.29 g orange product (64.22% yield). ¹H NMR (500 MHz, CDCl₃) δ 7.62 (dq, J=7.4, 3.0, 2.2 Hz, 2H), 7.42-7.35 (m, 3H), 7.27-7.22 (m, 2H), 7.22-7.17 (m, 3H), 4.39 (q, J=1.9 Hz, 1H), 4.27 (q, J=3.1, 2.7 Hz, 1H), 4.24-4.13 (m, 1H), 3.97 (s, 5H), 3.88 (d, J=2.4 Hz, 1H), 1.79 (s, 6H), 1.29 (d, J=6.7 Hz, 3H). ³¹P NMR (202 MHz, CDCl₃) δ-22.87.

### Example 2: Preparation of N,N-Dimethyl-(2-bis(4-fluorophenyl)phosphino)ferrocenylethylamine 2c (mixed)

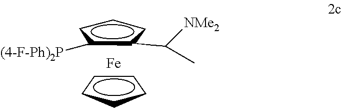

In a dry box, a 500 mL round bottom flask was charged with 4.26 g of racemic N,N-dimethyl-1-ferrocenylethylamine 1 (15.90 mmol) and 50 mL tert-butyl methyl ether (TBME). n-Butyllithium (7.63 mL, 19.08 mmol, 1.2 eq.) was added via an addition funnel over 1 hr while maintaining temperature below 25° C. The addition funnel was rinsed with 20 mL of TBME into the flask. The mixture was stirred at ambient temperature for 1 hr. Chlorobis(4-fluorophenyl)phosphine (5.00 g, 19.08 mmol, 1.2 eq.) in 20 mL TBME was added dropwise into the flask over 30 min, maintaining T<35° C. The mixture was stirred under reflux for 2.5 hrs and then at ambient temperature for 1.5 hrs. The mixture was cooled in an ice-water bath and quenched with 100 mL saturated aqueous sodium bicarbonate, while maintaining T below 15° C., followed by 150 mL water. The layers were separated, and the aqueous layer was extracted with toluene (3×200 mL). The combined organic layers were dried with sodium sulfate and concentrated to afford 9.57 g crude product as a brown oil. The crude product was crystallized from refluxing alcohol by cooling to 4° C. overnight. The precipitated, orange solid, was filtered off and washed with 50 mL cold ethanol at 0° C. yielding 5.29 g orange crude product. The crude product was used for next step. ¹H NMR (500 MHz, CDCl₃) δ 7.61-7.53 (m, 2H), 7.18 (m, 3H), 7.14-7.01 (m, 3H), 4.44-4.37 (m, 1H), 4.32-4.26 (m, 1H), 4.26-4.11 (q, 1H), 3.98 (s, 5H), 3.84-3.79 (m, 1H), 1.78 (s, 6H), 1.36-1.24 (m, 3H). ³¹P NMR (202 MHz, CDCl₃) δ-18.30.

### Example 3: Preparation of 2-(Diphenylphosphino)ferrocenyl]ethyldi-3,5-xylylphosphine 3b (mixed)

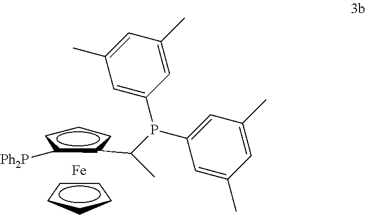

In a dry box, a 500 mL round bottom flask was charged with 4.65 g of mixed (N,N-dimethyl-(2-diphenylphosphino)ferrocenylethylamine 2b and 200 mL degassed acetic acid. Under N2 atmosphere, 2.95 mL of bis(3,5-dimethylphenyl)phosphine (12.14 mmol, 1.2 eq.) was slowly added to the flask, and the mixture was stirred at 120° C. for 1 hr. The mixture was cooled to ambient temperature and the solvent was removed under vacuum. Limiting air exposure, the residue was dissolved in 150 mL ethanol and refluxed for 2 hrs. The flask was cooled to ambient temperature and the solid product was filtered and washed with cold ethanol inside the purge box. to give 4.57 g orange solid (70.76% yield). ¹H NMR (500 MHz, CDCl₃) δ 7.68 (m, 2H), 7.44-7.38 (m, 3H), 7.19 (d, J=3.5 Hz, 5H), 6.96 (d, J=8.2 Hz, 3H), 6.91 (d, J=6.5 Hz, 2H), 6.79 (s, 1H), 4.27 (t, J=2.6 Hz, 1H), 4.05 (t, J=2.6 Hz, 2H), 3.87 (s, 5H), 3.74 (m, 1H), 2.29 (s, 6H), 2.21 (s, 6H), 1.49 (t, J=7.5 Hz, 3H). ³¹P NMR (202 MHz, CDCl₃) δ 6.90 (d, J=19.5 Hz), −24.84 (d, J=19.5 Hz).

### Example 4: Preparation of 2-(Diphenylphosphino)-ferrocenylethyldiphenylphosphine) 3c (mixed)

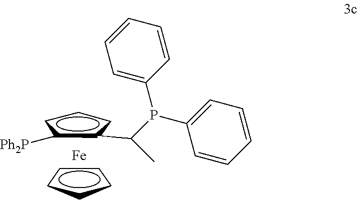

In a dry box, a 500 mL round bottom flask was charged with 15.00 g of mixed (N,N-dimethyl-(2-diphenylphosphino)ferrocenylethylamine 2b and 250 mL degassed acetic acid. Under N2 atmosphere, 6.95 mL of diphenylphosphine (39.15 mmol, 1.2 eq.) was slowly added to the flask, and the mixture was stirred at 120° C. for 1 hr. The mixture was cooled to ambient temperature and the solvent was removed under vacuum. Limiting air exposure, the residue was dissolved in 200 mL ethanol and refluxed for 2 hrs. The flask was cooled to ambient temperature and the solid product was filtered and washed with cold ethanol inside the purge box to give 14.65 g orange solid (77.09% yield). ¹H NMR (500 MHz, CDCl₃) δ 7.69 (m, 2H), 7.43-7.26 (m, 18H), 4.27 (t, J=2.6 Hz, 1H), 4.08 (m, 1H), 4.03 (m, 1H), 3.88 (s, 5H), 3.80 (m, 1H), 1.49 (t, J=7.3 Hz, 3H). ³¹P NMR (202 MHz, CDCl₃) δ 6.33 (d, J=21.0 Hz), −25.39 (d, J=21.0 Hz).

### Example 5: Preparation of (2-(Di-4-fluorophenylphosphino)-ferrocenylethyldiphenylphosphine) 3d (mixed)

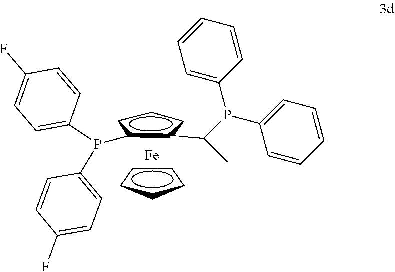

In a dry box, a 300 mL round bottom flask was charged with 5.34 g of mixed N,N-dimethyl-(2-bis(4-fluorophenyl)phosphino)ferrocenylethylamine 2c and 150 mL degassed acetic acid. Under N2 atmosphere, 1.79 mL diphenylphosphine (10.07 mmol, 1.2 eq.) was slowly added to the flask, and the mixture was stirred at 120° C. for 1 hr. The mixture was cooled to ambient temperature and the solvent was removed under vacuum. Limiting air exposure, the residue was dissolved in 150 mL ethanol and refluxed for 2 hrs. The flask was cooled to ambient temperature and the solid product was filtered and washed with cold ethanol inside the purge box to give 2.19 g orange solid (42.2% yield). ¹H NMR (500 MHz, CDCl₃) δ 7.64 (m, 2H), 7.40-67.11 (m, 14H), 6.94-6.88 (m, 2H), 4.30 (t, J=2.6 Hz, 1H), 4.04-3.98 (m, 2H), 3.90 (s, 5H), 3.83-3.71 (m, 1H), 1.45 (t, J=7.0 Hz, 3H). ³¹P NMR (202 MHz, CDCl₃) δ 7.05 (d, J=22.4 Hz), −27.85 (dd, J=22.2, 4.2 Hz).

### Example 6: Preparation of (2-(Diphenylphosphino)ferrocenyl]ethyldi-4-fluorophenylphosphine) 3e (mixed)

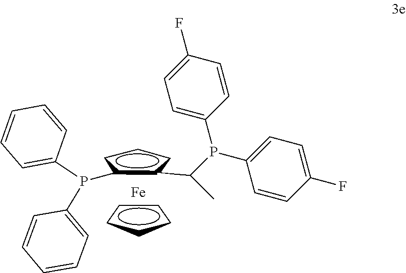

In a dry box, a 300 mL round bottom flask was charged with 6.94 g of mixed (N,N-dimethyl-(2-diphenylphosphino)ferrocenylethylamine 2b, 5.75 g of bis(4-fluorophenyl)phosphine (18.12 mmol, 1.2 eq.) and 150 mL degassed acetic acid. The mixture was stirred at 120° C. for 1 hr. The mixture was cooled to ambient temperature and the solvent was removed under vacuum. Limiting air exposure, the residue was dissolved in 200 mL ethanol and refluxed for 2 hrs. The flask was cooled to ambient temperature and the solid product was filtered and washed with cold ethanol inside the purge box to give 7.89 g orange solid (84.51% yield). ¹H NMR (500 MHz, CDCl₃) δ 7.67 (m, 2H), 7.42-7.17 (m, 12H), 7.03 (m, 2H), 6.87 (m, 2H), 4.30 (t, J=2.6 Hz, 1H), 4.12-4.06 (m, 2H), 3.87 (s, 5H), 3.75 (m, 1H), 1.48 (dd, J=8.7, 7.1 Hz, 3H). ³¹P NMR (202 MHz, CDCl₃) δ 3.55 (dt, J=18.8, 4.8 Hz), −26.04 (d, J=18.8 Hz).

### Example 7: (2-(Di-4-fluorophenylphosphino)ferrocenyl ethyl di-4-flurophenylphosphine) 3f (mixed)

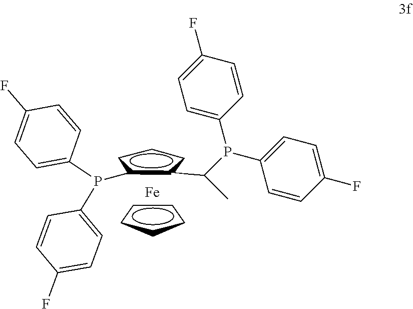

In a dry box, a 300 mL round bottom flask was charged with 7.57 g of mixed N,N-dimethyl-(2-bis(4-fluorophenyl)phosphino)ferrocenylethylamine 2c, 4.83 of g bis(4-fluorophenyl)phosphine (15.23 mmol, 1.2 eq.) and 150 mL degassed acetic acid. The mixture was stirred at 120° C. for 1 hr. The mixture was cooled to ambient temperature and the solvent was removed under vacuum. Limiting air exposure, the residue was dissolved in 200 mL ethanol and refluxed for 2 hrs. The flask was cooled to ambient temperature and the solid product was filtered and washed with cold ethanol inside the purge box to 1.72 g orange solid (45.72% yield). ¹H NMR (500 MHz, CDCl₃) δ 7.62 (td, J=7.9, 5.7 Hz, 2H), 7.24-7.01 (m, 10H), 6.91 (m, 4H), 4.33 (t, J=2.6 Hz, 1H), 4.07-4.01 (m, 2H), 3.91 (s, 5H), 3.73 (m, 3.9 Hz, 1H), 1.4ff5 (t, J=7.5 Hz, 3H). ³¹P NMR (202 MHz, CDCl₃) δ 4.49 (dd, J=20.6, 4.1 Hz), −28.17 (dd, J=4.3 Hz).

### Example 8: Hydroformylation Process, Set-Up, and Catalyst Preparation

Propylene was reacted with hydrogen and carbon monoxide in a vapor take-off reactor made of a vertically arranged stainless steel pipe having a 2.5 cm inside diameter and a length of 1.2 meters to produce butyraldehydes. The reactor was encased in an external jacket that was connected to a hot oil machine. The reactor had a filter element located 35 cm in the side near the bottom of the reactor for the inlet of gaseous reactants. The reactor contained a thermocouple, which was arranged axially with the reactor in its center for accurate measurement of the temperature of the hydroformylation reaction mixture. The bottom of the reactor had a high-pressure tubing connection that was connected to a cross. One of the connections to the cross permitted the addition of non-gaseous reactants (such as higher boiling alkenes or make-up solvents), another led to the high-pressure connection of a differential pressure (D/P) cell that was used to measure catalyst level in the reactor, and the bottom connection was used for draining the catalyst solution at the end of the run.

In the hydroformylation of propylene in a vapor take-off mode of operation, the hydroformylation reaction mixture or solution containing the catalyst was sparged under pressure with the incoming reactants of propylene, hydrogen, and carbon monoxide as well as any inert feed, such as nitrogen. As butyraldehyde was formed in the catalyst solution, it and unreacted reactant gases were removed as a vapor from the top of the reactor by a side-port. The removed vapor was chilled in a high-pressure separator where the butyraldehyde product was condensed along with some of the unreacted propylene. The uncondensed gases were let down to atmospheric pressure via the pressure control valve. The product from the high-pressure separator was subsequently weighed and analyzed by standard gas/liquid phase chromatography (GC/LC) techniques for the net weight and normal/iso ratio of the butyraldehyde product. Activity was calculated as pounds of butyraldehydes produced per gram of rhodium per hour (lbs. HBu/gr-Rh-hr).

The gaseous feeds were introduced into the reactor via twin cylinder manifolds and high-pressure regulators. The hydrogen passed through a mass flow controller and then through a commercially available “Deoxo” (registered trademark of Engelhard Inc.) catalyst bed to remove any oxygen contamination. The carbon monoxide passed through an iron carbonyl removal bed (as disclosed in U.S. Pat. No. 4,608,239), a similar “Deoxo” bed heated to 125° C., and then a mass flow controller.

Nitrogen can be added to the feed mixture as an inert gas. Nitrogen, when added, was metered in and then mixed with the hydrogen feed prior to the hydrogen Deoxo bed. Propylene was fed to the reactor from feed tanks that were pressurized with nitrogen and was controlled using a liquid mass flow meter. All gases and propylene were passed through a preheater to ensure complete vaporization of the liquid propylene prior to entering the reactor.

A catalyst solution was prepared under nitrogen using a charge of 18.9 milligrams of dicarbonylacetylacetonato rhodium(I) and various amounts of the ligands as indicated in Tables 1-8, 190 mL of dioctylterephthalate (DOTP) or 190 mL of dodecane and 40 mL of toluene. The mixture was stirred under nitrogen (and heated, if necessary) until a homogeneous solution was obtained. The mixture was charged to the reactor in a manner described previously, and the reactor was sealed. The reactor pressure control was set at 17.9 bar (260 psig), and the external oil jacket on the reactor was heated to the desired temperature. Hydrogen, carbon monoxide, nitrogen, and propylene vapors were fed through the frit at the base of the reactor, and the reactor was allowed to build pressure. The propylene was metered as a liquid and fed at a rate specified in Tables 1 to 8. The temperature of the external oil was modified to maintain an internal reactor temperature of desired value. The unit was usually operated for 5 hours, and hourly samples were taken. The hourly samples were analyzed as described above using a standard GC method. The last three samples of the run were used to determine the N/I ratio and catalyst activity.

The results of the daylong bench unit runs are summarized in Tables 1-8

The results in Tables 1 and 2 for the stereospecific isomer 3a at different temperatures using two different solvents.

TABLE 1
Hydroformylation of propylene using ligand 3a.

| | | | | | | Propylene | Total Feed | | | Activity |
|---|---|---|---|---|---|---|---|---|---|---|
| Temp | | | | | RhAcAc(CO)2 | Setpoint | Pressure, | | | (lb HBu/g |
| (deg C) | Ligand | Solvent | Exp | L:Rh | Loading (mg) | (g/hr) | psig | N/I | % iHBu | Rh-hr) |
| 115 | 3a | dodecane | 1 | 4 | 18.9 | 356 | 260.0 | 0.55 | 64.5 | 2.30 |
| 115 | 3a | dodecane | 2 | 4 | 18.9 | 450 | 260.0 | 0.55 | 64.5 | 3.23 |
| 115 | 3a | dodecane | 3 | 4 | 18.9 | 520 | 260.0 | 0.55 | 64.5 | 3.51 |
| 115 | 3a | DOTP | 4 | 2 | 18.9 | 520 | 260.0 | 1.22 | 45.0 | 6.81* |
| 115 | 3a | DOTP | 5 | 3 | 18.9 | 520 | 260.0 | 0.54 | 64.9 | 2.83 |

*Activity still increases after 5 h.

TABLE 2
Hydroformylation of propylene using ligand 3a.

| | | | | | | Propylene | Total Feed | | | Activity |
|---|---|---|---|---|---|---|---|---|---|---|
| Temp | | | | | RhAcAc(CO)2 | Setpoint | Pressure, | | | (lb HBu/g |
| (deg C) | Ligand | Solvent | Exp | L:Rh | Loading (mg) | (g/hr) | psig | N/I | % iHBu | Rh-hr) |
| 105 | 3a | dodecane | 6 | 4 | 18.9 | 356 | 260.0 | 0.57 | 63.7 | 0.69 |
| 115 | 3a | dodecane | 7 | 4 | 18.9 | 356 | 260.0 | 0.55 | 64.5 | 2.30 |
| 120 | 3a | dodecane | 8 | 4 | 18.9 | 356 | 260.0 | 0.58 | 63.3 | 3.80 |
| 125 | 3a | dodecane | 9 | 4 | 18.9 | 356 | 260.0 | 0.60 | 62.5 | 4.52 |

TABLE 3
Hydroformylation of propylene using ligand 3b.

| | | | | | | Propylene | Total Feed | | | Activity |
|---|---|---|---|---|---|---|---|---|---|---|
| Temp | | | | | RhAcAc(CO)2 | Setpoint | Pressure, | | | (lb HBu/g |
| (deg C) | Ligand | Solvent | Exp | L:Rh | Loading (mg) | (g/hr) | psig | N/I | % iHBu | Rh-hr) |
| 115 | 3b | DOTP | 10 | 4 | 18.9 | 520 | 260.0 | 0.55 | 64.5 | 2.97 |
| 115 | 3b | DOTP | 11 | 4 | 18.9 | 520 | 260.0 | 0.53 | 65.4 | 3.08 |
| 115 | 3b | dodecane | 12 | 4 | 18.9 | 520 | 260.0 | 0.54 | 64.9 | 3.94 |
| 115 | 3b | DOTP | 13 | 2 | 18.9 | 520 | 260.0 | 1.27 | 44.1 | 9.71* |
| 115 | 3b | DOTP | 14 | 6 | 18.9 | 520 | 260.0 | 0.53 | 65.4 | 3.04 |

*Activity still increases after 5 h.

The results in table 3 are for the inventive mixture 3b. We note that 3a and 3b give similar isoselectivity, allowing use of the more economical mixture made using the inventive process of the invention.

TABLE 4
Hydroformylation of propylene using ligand 3c.

| | | | | | | Propylene | Total Feed | | | Activity |
|---|---|---|---|---|---|---|---|---|---|---|
| Temp | | | | | RhAcAc(CO)2 | Setpoint | Pressure, | | | (lb HBu/g |
| (deg C) | Ligand | Solvent | Exp | L:Rh | Loading (mg) | (g/hr) | psig | N/I | % iHBu | Rh-hr) |
| 115 | 3c | DOTP | 15 | 4 | 18.9 | 356 | 260.0 | 0.61 | 62.1 | 2.11 |
| 115 | 3c | DOTP | 16 | 2 | 18.9 | 356 | 260.0 | 1.24 | 44.6 | 6.51* |
| 115 | 3c | DOTP | 17 | 1 | 18.9 | 356 | 260.0 | 1.25 | 44.4 | 6.52* |
| 115 | 3c | dodecane | 18 | 4 | 18.9 | 356 | 260.0 | 0.58 | 63.3 | 3.61 |

*Activity still increases after 5 h.

The results in tables 4 and 5 are for the inventive mixture 3c.

TABLE 5
Hydroformylation of propylene using ligand 3c.

| | | | | | | Propylene | Total Feed | | | Activity |
|---|---|---|---|---|---|---|---|---|---|---|
| Temp | | | | | RhAcAc(CO)2 | Setpoint | Pressure, | | | (lb HBu/g |
| (deg C) | Ligand | Solvent | Exp | L:Rh | Loading (mg) | (g/hr) | psig | N/I | % iHBu | Rh-hr) |
| 115 | 3c | DOTP | 19 | 4 | 18.9 | 356 | 260.0 | 0.61 | 62.1 | 2.11 |
| 115 | 3c | DOTP | 20 | 4 | 18.9 | 520 | 260.0 | 0.55 | 64.5 | 3.24 |
| 115 | 3c | DOTP | 21 | 4 | 18.9 | 600 | 260.0 | 0.57 | 63.7 | 6.71 |
| 115 | 3c | DOTP | 22 | 4 | 18.9 | 600 | 260.0 | 0.56 | 64.1 | 6.42 |

TABLE 6
Hydroformylation of propylene using ligand 3d.

| | | | | | | Propylene | Total Feed | | | Activity |
|---|---|---|---|---|---|---|---|---|---|---|
| Temp | | | | | RhAcAc(CO)2 | Setpoint | Pressure, | | | (lb HBu/g |
| (deg C) | Ligand | Solvent | Exp | L:Rh | Loading (mg) | (g/hr) | psig | N/I | % iHBu | Rh-hr) |
| 115 | 3d | DOTP | 23 | 4 | 18.9 | 520 | 260.0 | 0.56 | 64.1 | 4.69 |
| 115 | 3d | DOTP | 24 | 6 | 18.9 | 520 | 260.0 | 0.56 | 64.1 | 4.25 |
| 115 | 3d | DOTP | 25 | 2 | 18.9 | 520 | 260.0 | 1.19 | 45.7 | 5.2* |
| 115 | 3d | dodecane | 26 | 4 | 18.9 | 520 | 260.0 | 0.55 | 64.5 | 3.8 |
| 115 | 3d | DOTP | 27 | 4 | 18.9 | 600 | 260.0 | 0.56 | 64.1 | 4.96 |

*Activity still increases after 5 h.

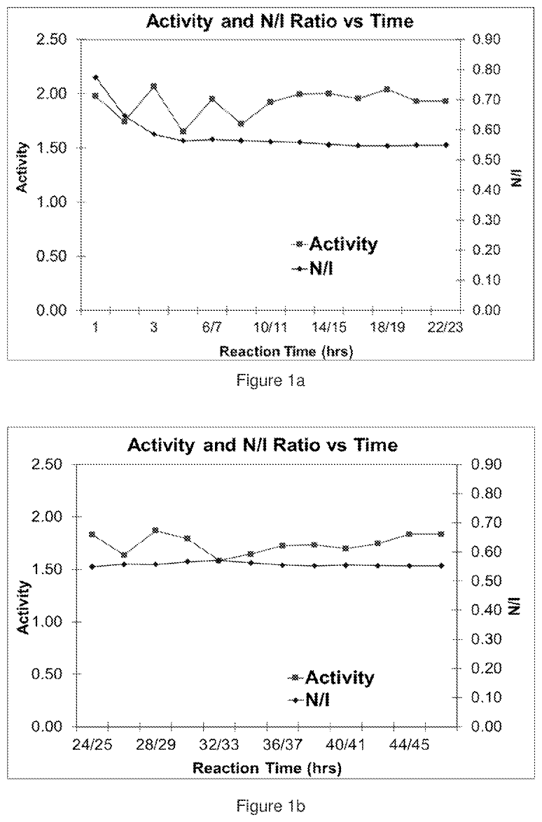

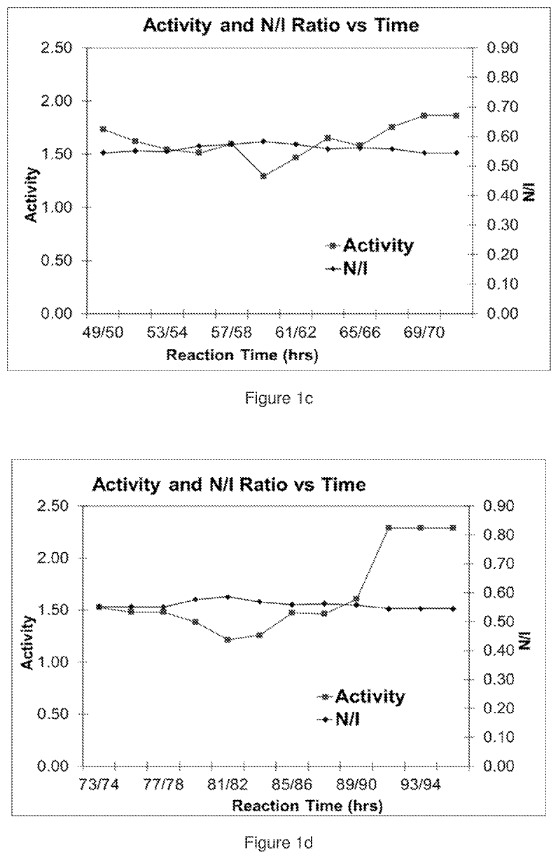

FIGS. 1a to 1d demonstrate the results of 4 days' continuous hydroformylation of propylene using ligand 3d, under the following conditions: 110 C, L/Rh=6, DOTP solvent, propylene flow (4.18 SLPM), syn gas flow (3.4 SLPM), Nitrogen gas flow (1.66 SLPM). Activity is expressed in lbs RHCO/g Rh/hr). FIG. 1a is Day 1, FIG. 1b is Day 2; FIG. 1c is Day 3, and FIG. 1d is Day 4. This indicates the thermal stability of the inventive ligands of the invention.

TABLE 7
Hydroformylation of propylene using ligand 3e.

| | | | | | | Propylene | Total Feed | | | Activity |
|---|---|---|---|---|---|---|---|---|---|---|
| Temp | | | | | RhAcAc(CO)2 | Setpoint | Pressure, | | | (lb HBu/g |
| (deg C) | Ligand | Solvent | Exp | L:Rh | Loading (mg) | (g/hr) | psi | N/I | % iHBu | Rh-hr) |
| 115 | 3e | DOTP | 28 | 4 | 18.9 | 520 | 260.0 | 0.58 | 63.3 | 4.77 |
| 115 | 3e | DOTP | 29 | 4 | 18.9 | 520 | 260.0 | 0.57 | 63.7 | 4.42 |
| 115 | 3e | DOTP | 30 | 6 | 18.9 | 520 | 260.0 | 0.56 | 64.1 | 5.04 |
| 115 | 3e | DOTP | 31 | 2 | 18.9 | 520 | 260.0 | 1.12 | 47.2 | 9.12* |

*Activity still increases after 5 h.

TABLE 8
Hydroformylation of propylene using ligand 3f.

| | | | | | | Propylene | Total Feed | | | Activity |
|---|---|---|---|---|---|---|---|---|---|---|
| Temp | | | | | RhAcAc(CO)2 | Setpoint | Pressure, | | | (lb HBu/g |
| (deg C) | Ligand | Solvent | Exp | L:Rh | Loading (mg) | (g/hr) | psig | N/I | % iHBu | Rh-hr) |
| 115 | 3f | DOTP | 32 | 4 | 18.9 | 520 | 260.0 | 0.56 | 64.1 | 7.2 |
| 115 | 3f | DOTP | 33 | 4 | 18.9 | 520 | 260.0 | 0.56 | 64.1 | 7.4 |
| 115 | 3f | DOTP | 34 | 2 | 18.9 | 520 | 260.0 | 1.17 | 46.1 | 8.94* |

*Activity still increases after 5 h.

We claim:

1. A process for preparing at least one aldehyde product under hydroformylation temperature and pressure conditions, comprising contacting at least one olefin with hydrogen and carbon monoxide in the presence of at least one solvent and a transition metal-based catalyst composition comprising a ferrocene-based biphosphine ligand represented by the following general formula A and its mirror images: 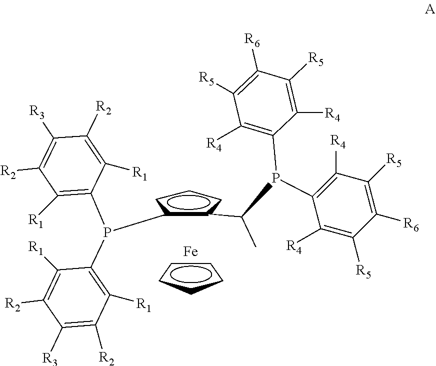
wherein:
R1, R2, R3, R4, R5 and R6 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent.

2. The process of claim 1, wherein the transition metal-based catalyst composition further comprises a mixture of ferrocene-based biphosphine ligands represented by the following general formulas A and B and its mirror images: 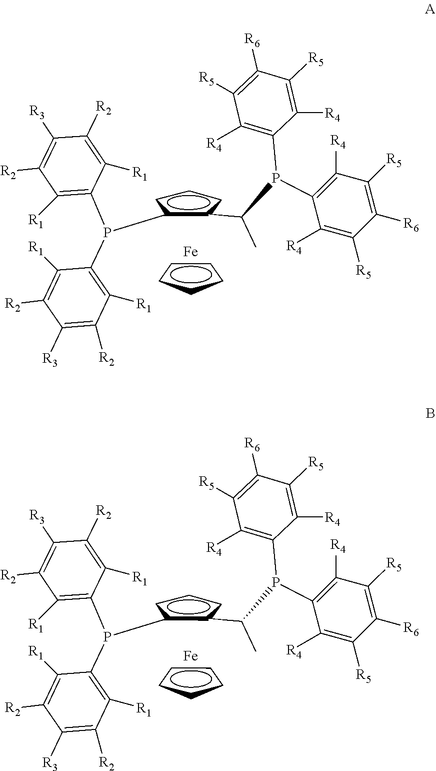
wherein:
R1, R2, R3, R4, R5 and R6 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent.

3. The process of claim 1, wherein R1, R2, R3, R4, R5 and R6 may be independently selected from H or methyl.

4. The process of claim 1, wherein one or more of R3 and R6 may be independently selected from F, Cl, or Br.

5. The process of claim 2, wherein R1, R2, R3, R4, R5 and R6 may be independently selected from H or methyl.

6. The process of claim 2, wherein one or more of R3 and R6 may be independently selected from F, Cl, or Br.

7. The process of claim 2, wherein R1, R2, R3, R4, R5 and R6 are H.

8. The process of claim 1, wherein the ferrocene-based biphosphine ligand comprises a stereoisomer or mixture of one or more of:

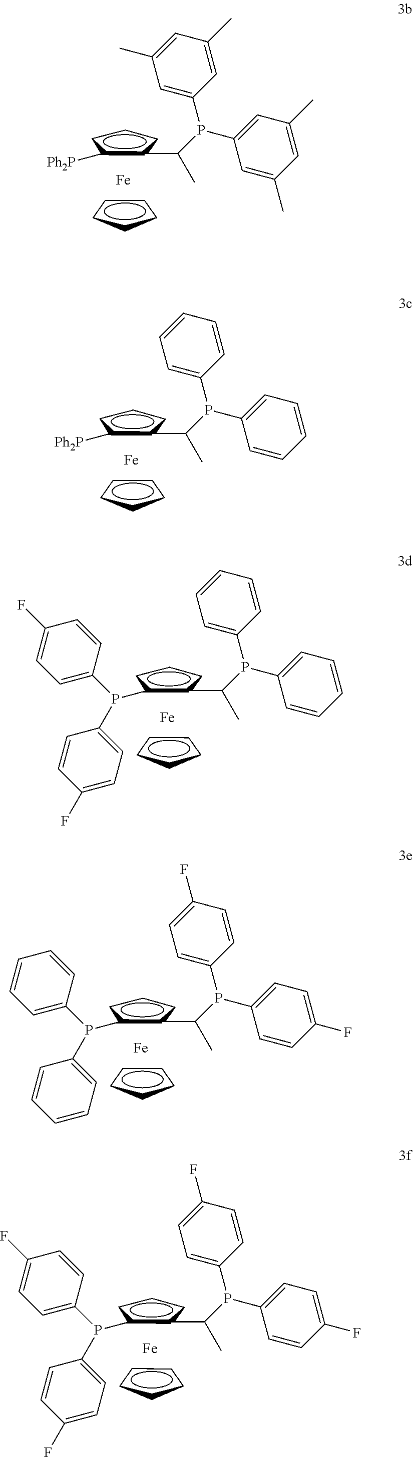

9. The process of claim 1, wherein the ferrocene-based biphosphine ligand comprises a stereoisomer or mixture of:

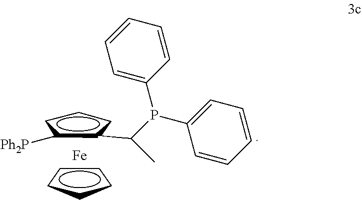

10. The process of claim 1, wherein the transition metal-based catalyst composition comprises rhodium.

11. The process of claim 8, wherein the ratio of ligand to rhodium is from 1:1 to 50:1, based on the molar ratio of ligand to rhodium.

12. The process of claim 8, wherein the ratio of ligand to rhodium is from 2:1 to 40, based on the molar ratio of ligand to rhodium.

13. The process of claim 1, wherein the at least one olefin comprises propylene and the at least one aldehyde product comprises a mixture of iso-butyraldehyde and n-butyraldehyde.

14. The process of claim 1, wherein the aldehyde product of the process comprises an iso-selectivity of about 55% to about 70%.

15. The process of claim 1, wherein the aldehyde product of the process comprises an iso-selectivity of about 56% to about 68%.

16. The process of claim 1, wherein the aldehyde product of the process comprises an iso-selectivity of 55% or greater.

17. The process of claim 1, wherein the pressure ranges from about 2 atm to about 80 atm.

18. The process of claim 1, wherein the temperature ranges from about 40 about 120 degrees Celsius.

19. The process of claim 1, wherein the temperature is at least 80 degrees Celsius.
